(19) United States
(12) Patent Application Publication
(10) Pub. No.: US 2023/0329950 A1
(43) Pub. Date: Oct. 19, 2023

(54) Operating Table Cover Having Fast Recovery Foam

(71) Applicant: Prime Medical, LLC, Knoxville, TN (US)

(72) Inventor: David Holladay, Knoxville, TN (US)

(21) Appl. No.: 18/296,155

(22) Filed: Apr. 5, 2023

Related U.S. Application Data
(60) Provisional application No. 63/362,893, filed on Apr. 13, 2022.

(51) Int. Cl. A61G 13/10

(52) U.S. Cl. CPC A61G 13/10

(57) ABSTRACT

Various implementations include an operating table anti-skid pad. The pad includes a body having a longitudinal axis, a first edge, and a second edge spaced apart and opposite from the first edge. The body further has a third edge extending between the first edge and the second edge and a fourth edge opposite and spaced apart from the third edge. The body includes non-viscoelastic, resiliently deformable material having a rate of recovery of 2.0 seconds or less.

### FIELD

This disclosure relates to the field of operating room anti-skid pads. More particularly, this disclosure relates to an operating room anti-skid pad formed of a fast recovery foam to prevent sliding of a patient on an operating table.

### BACKGROUND

Surface covers for operating tables may be used for various medical procedures, such as to prevent sliding of a patient on the operating table. For example, an operating table overlay may be used when a patient is to be positioned in the Trendelenburg position, wherein a head of the patient is declined below the feet of the patient at an angle of about 15-45 degrees. It may be desirable for the operating table cover to aid in preventing sliding of the patient when in the Trendelenburg position or other positions on the operating table.

Existing surface covers may be formed of a viscoelastic foam. The properties of viscoelastic foam in relation to an operating table cover or pad may create many unsuitable characteristics. For example, using viscoelastic foam with a slow rate of recovery may make transportation and packaging of an operating table cover difficult due to the viscoelastic foam not fully recovering or requiring a significant period of time to recover a desired shape. Further, characteristics of an operating table pad or cover formed of viscoelastic foam may vary depending on a temperature of the foam. Viscoelastic foam may have a relatively narrow range at which the viscoelastic foam maintains desirable deformability characteristics. Viscoelastic foam may have other undesirable characteristics, such as retaining heat of a patient laying on the viscoelastic foam and thereby causing the patient’s skin to become elevated, which is undesirable due to the risk of decubitus (pressure) ulcer formation cause by factors that include increased temperature and moisture.

Other traditional foams, such as traditional polyurethane foam, may only be suitable for use with the aid of table straps or other fasteners to prevent sliding or movement of a pad on an operating surface.

### SUMMARY

Various implementations include an operating table anti-skid pad. The pad includes a body having a longitudinal axis, a first edge, and a second edge spaced apart and opposite from the first edge. The body further has a third edge extending between the first edge and the second edge and a fourth edge opposite and spaced apart from the third edge. The body includes non-viscoelastic, resiliently deformable material having a rate of recovery of 2.0 seconds or less.

In some implementations, the rate of recovery is 1.5 seconds or less. In some implementations, the rate of recovery is 1.0 seconds or less.

In some implementations, the material includes a polyol. In some implementations, the polyol includes a high molecular weight polyol. In some implementations, the material is a non-viscoelastic foam.

In some implementations, the body has a glass transition temperature of less than -20° C. In some implementations, the glass transition temperature is less than -50° C.

In some implementations, a static coefficient of friction of the material is 0.9 or greater. In some implementations, the static coefficient of the material is 1.0 or greater. In some implementations, the static coefficient of friction of the material is 1.1 or greater.

In some implementations, the body further has a first surface extending between the first edge, the second edge, the third edge, and the fourth edge and a second surface opposite and spaced apart from the first surface. In some implementations, the body has a thickness as measured between the first surface and the second surface. In some implementations, the thickness of the body is 4 inches or less. In some implementations, the thickness is from about 1 inch to about 2 inches. In some implementations, the body has a uniform the thickness. In some implementations, the body has a variable the thickness.

In some implementations, the body has a first surface extending between the first edge, the second edge, the third edge, and the fourth edge and a second surface opposite and spaced apart from the first surface. In some implementations, the first surface of the body defines a cutout adjacent the first edge and extending toward the second surface. In some implementations, the cutout extends to the second surface. In some implementations, the cutout is at least partially defined by the first edge. In some implementations, the cutout is semi-circular or circular as viewed in a plane defined by the first surface.

In some implementations, the pad further includes one or more straps extending from the third edge or the fourth edge. In some implementations, the one or more straps each include a fastener for coupling the strap to a rail of an operating table. In some implementations, the fastener includes a hook and loop fastener. In some implementations, the straps do not include a fastener.

In some implementations, the pad further includes one or more arm tucking wings extending from the third edge or the fourth edge. In some implementations, the one or more arm tucking wings are disposed closer to the first edge than to the second edge.

Various other implementations include a system including a pad as described herein and a vacuum sealed package. In some implementations, the pad is disposed within the vacuum sealed package. In some implementations, the pad is compressed by a negative pressure within the vacuum sealed package.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows a perspective view of an operating table anti-skid pad according to one embodiment of the present disclosure;

FIG. 2 shows a perspective view of an operating table anti-skid pad having table rail straps according to one embodiment of the present disclosure;

FIG. 3 shows a perspective view of an operating table anti-skid pad having arm tucking and protection wings according to one embodiment of the present disclosure;

FIG. 4 shows a detail view of a non-viscoelastic foam of an operating table anti-skid pad according to any one of the embodiments of the present disclosure; and

FIG. 5 shows a perspective view of an operating table anti-skid pad according to any one of the embodiments of the present disclosure that has been rolled or compressed.

### DETAILED DESCRIPTION

Various terms used herein are intended to have particular meanings. Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

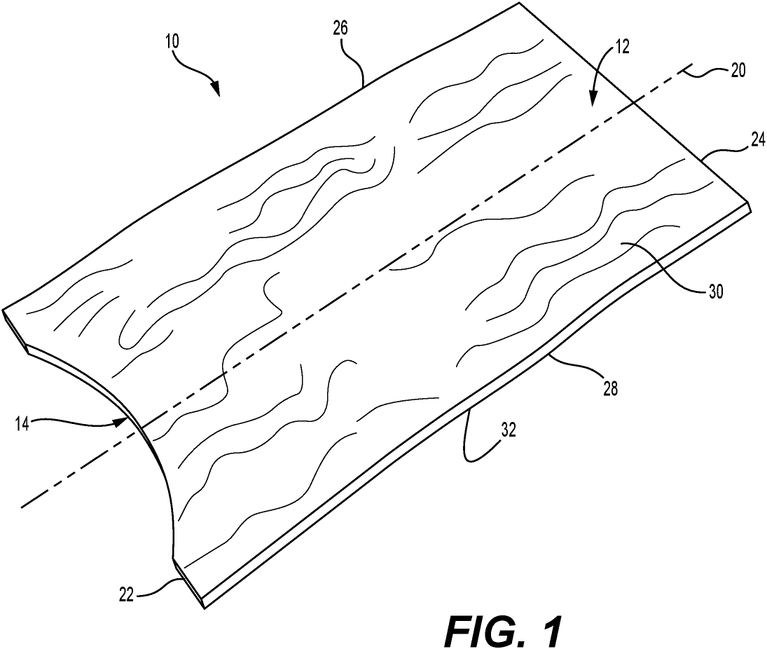

Embodiments herein relate to an operating table anti-skid pad 10, such as the operating table anti-skid pad 10 shown in FIG. 1, that is formed with a non-viscoelastic foam, such as non-viscoelastic polyurethane foam. The operating table anti-skid pad 10 may be substantially formed of a non-viscoelastic foam that has a coefficient of friction that is suitable to substantially prevent sliding of a patient on an operating table or other surface, particularly during a procedure in which the patient may be oriented at an incline or angle. For example, embodiments of the operating table anti-skid pad 10 disclosed herein may be suitable for use when a patient is to be maintained in the Trendelenburg position.

A suitable foam for use in the operating table anti-skid pad 10 described herein is a non-viscoelastic foam having a relatively high rate of recovery or the time at which the non-viscoelastic foam returns to its starting shape after being deformed. For example, a suitable material for the operating table anti-skid pad 10 may have a rate of recovery that is substantially faster than a rate of recovery of a viscoelastic foam. Where a viscoelastic foam material may have a recovery time of slower than two seconds, the operating table cover 10 may have a rate of recovery that is substantially faster. A rate of recovery of a suitable foam for the operating table anti-skid pad 10 described herein may be one and a half seconds or less or one second or less, and in some embodiments may be substantially less than one second. For example, a rate of recovery may be high enough such that a measurement of the rate of recovery may be indeterminate or instantaneous due to the rapid rate at which the foam recovers to an original shape after being deformed. For instances in which the rate of recovery is indeterminate or instantaneous, the material recovers at the same speed as the indenter foot is removed after indentation such that contact between the material and the indenter foot is maintained.

The non-viscoelastic foam may be formed from an active response polyurethane or air infused polyurethane comprised of a combination of toluene diisocyanate (TDI) and a high molecular weight polyol, such as those with a molecular weight from 2000 to 10,000, thereby allowing the operating table anti-skid pad 10 to be substantially pliable at a wide range of temperatures including low temperatures. A glass transition temperature of a suitable non-viscoelastic foam, such as a non-viscoelastic polyurethane foam, may be substantially less than a glass transition temperature of a viscoelastic foam. For example, a suitable non-viscoelastic foam may have a glass transition temperature of -20° C. or less and may be approximately -66° C. or less. A low glass transition temperature of a suitable non-viscoelastic foam allows the operating table anti-skid pad 10 to remain substantially pliable at a wider range of ambient temperatures relative to a viscoelastic foam.

The non-viscoelastic foam forming the operating table anti-skid pad 10 may be resiliently deformable such that the non-viscoelastic foam may deform around a contour of a patient positioned on the operating table anti-skid pad 10 while retaining a high rebound rate to a non-deformed shape. Embodiments of the operating table anti-skid pad 10 described herein may provide substantially instantaneous customized support to a shape of a patient laying thereon. Further, if a position of a patient shifts, the operating table anti-skid pad 10 may substantially instantaneously respond to such shifting such that the operating table anti-skid pad 10 may rapidly contour to the patient in a new position of the patient.

The non-viscoelastic foam may be formed having air capsules formed therein, such as microscopic air capsules formed within the non-viscoelastic foam that may be created during manufacturing of the non-viscoelastic foam.

A suitable non-viscoelastic foam may further have suitable anti-skid properties to prevent sliding of a patient on an operating table. The non-viscoelastic foam may have a suitable coefficient of friction such that a patient does not slide along an operating table or other surface on which the operating table anti-skid pad 10 is located. In particular, when a patient is laying on the operating table anti-skid pad 10, the non-viscoelastic foam may be deformable and may interact with a surface of the operating table to prevent sliding of the patient and the operating table anti-skid pad 10 on the operating table, even when the patient is at least partially inclined or declined on the operating table. A suitable coefficient of friction of the non-viscoelastic foam is such that the operating table anti-skid pad 10 does not slide or otherwise substantially move against a surface of an operating table on which the operating table anti-skid pad 10 is located, particularly when the operating table is at least partially inclined or declined with a patient thereon during a procedure. Further, embodiments of the operating table anti-skid pad herein may be such that a coefficient of friction of the non-viscoelastic foam substantially prevents sliding or movement of the operating table anti-skid pad 10 on an operating table without requiring use of straps, tape, or other mechanisms required to prevent substantial sliding or movement.

FIG. 1 shows a basic embodiment of an example of the operating table anti-skid pad 10 for aiding in positioning of a patient on an operating table. The pad includes a body 12, two straps, and two arm tucking wings.

The operating table anti-skid pad 10 is formed of a foam material as described in greater detail herein that advantageously aids in preventing sliding of a patient on an operating table, such as when the patient is in the Trendelenburg position. The operating table anti-skid pad 10 is formed of a non-viscoelastic foam that is deformable with a fast rate of recovery, particularly in relation to a recovery rate of viscoelastic foams. The operating table anti-skid pad 10 substantially prevents or reduces sliding of a patient on the operating table and to provide improved pressure mapping across the operating table anti-skid pad 10 when supporting a patient thereon.

The body 12 has a longitudinal axis 20, a first edge 22, a second edge 24 spaced apart and opposite from the first edge 22, a third edge 26 extending between the first edge 22 and the second edge 24, and a fourth edge 28 opposite and spaced apart from the third edge 26. The body 12 further has a first surface 30 extending between the first edge 22, the second edge 24, the third edge 26, and the fourth edge 28 and a second surface 32 opposite and spaced apart from the first surface 30.

The body 12 may be formed having a thickness, as measured between the first surface 30 and the second surface 32, of from about 1 inch to about 2 inches, and may be as thick as 4 inches. A thickness of the body 12 may be substantially uniform. Alternatively, a thickness of the body 12 may vary across the body 12. For example, the body 12 of the pad 10 may have a thickness that tapers from the first end 22 to the second end 24 or may have a thicker portion where the patient’s head is intended to be placed during use.

The first surface 30 of the body 12 of the pad 10 may define a cutout 14 adjacent the first edge 22 and extending toward the second surface 32. The cutout 14 shown in FIGS. 1-3 extends to the second surface 32, but in some implementations, the cutout may extend only partially through the body from the first surface toward the second surface. In some implementations, the body defines two or more cutouts. In some implementations, the cutout is located adjacent any edge of the body or spaced apart from the edges. The cutout 14 may be utilized for procedures wherein access to a lower portion of a body of a patient may be desirable. The cutout 14 may be semi-circular as viewed in a plane defined by the first surface 30, or the cutout 14 may be formed in other various suitable shapes depending on a desired use of the operating table anti-skid pad 10.

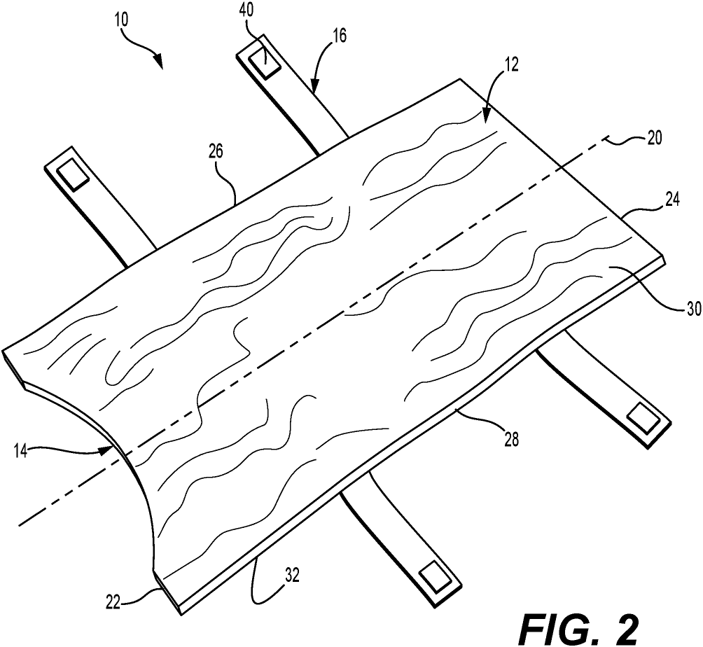

Referring to FIG. 2, the operating table anti-skid pad 10 may include one or more table rail straps 16. The one or more table rail straps 16 may coupled to or coupled adjacent to the third edge 26 and/or fourth edge 28 of the body 12 of the operating table anti-skid pad 10 such that the straps 16 are extendable from the third edge 26 and/or the fourth edge 28. The straps 16 may be positioned such that the straps 16 may further secure the operating table anti-skid pad 10 to an operating table, such as to railings of the operating table.

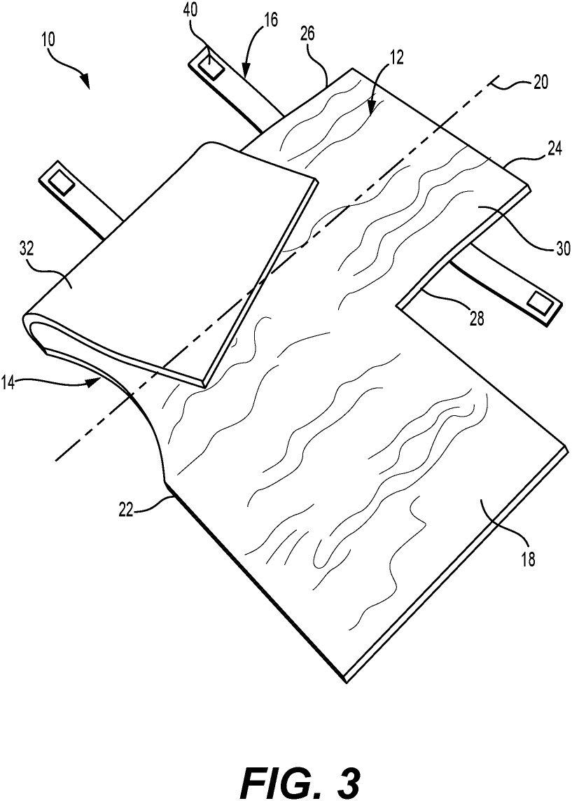

The one or more table rail straps 16 may include fasteners 40 formed thereon, such as hook and loop fasteners for securing the one or more table rail straps 16 to the operating table. The one or more table rail straps 16 may be used to secure the operating table anti-skid pad 10 in place on an operating surface, such as an operating table, such as when transferring a patient from a stretcher to the operating table or from the operating table to a stretcher. Although the straps 16 shown in FIGS. 2 and 3 include hook and loop fasteners 40, in some implementations, the straps include any other type of fastener, such as snaps, buttons, eyelets, threaded fasteners such as screws, bolts, nuts, or zippers, notches, barbs, clamps, friction coating, pins, buckles, magnets, compression/friction/interference fittings, ties, zip ties, adhesives, or any other type of fastener known in the art capable of coupling the strap to an operating table. In some implementations, the straps do not include fasteners. In some implementations, the straps can be wrapped around a portion of the operating table, and the static friction of the material of the body of the pad is great enough such that the ends of the straps can be disposed under the second surface of the body of the pad and the force of the patient on disposed on the first surface of the body of the pad secures the ends of the straps to the second surface.

As shown in FIG. 3, the operating table anti-skid pad 10 may further include one or more arm tucking wings 18. The one or more arm tucking wings 18 may extend from the third edge 26 and/or the fourth edge 28 and be located closer to the first edge 22 than to the second edge 24 of the operating table anti-skid pad 10, proximate to the cutout 14 formed on the operating table anti-skid pad 10. The one or more arm tucking wings 18 may extend laterally from third edge 26 and/or fourth edge 28 of the body 12 of the operating table anti-skid pad 10 and may be positioned adjacent arms of a patient when the patient is on the operating table anti-skid pad 10. The operating table anti-skid pad 10 may further include other accessories, including but not limited to body straps, disposable draw sheets, head cradles, and arm sleeves.

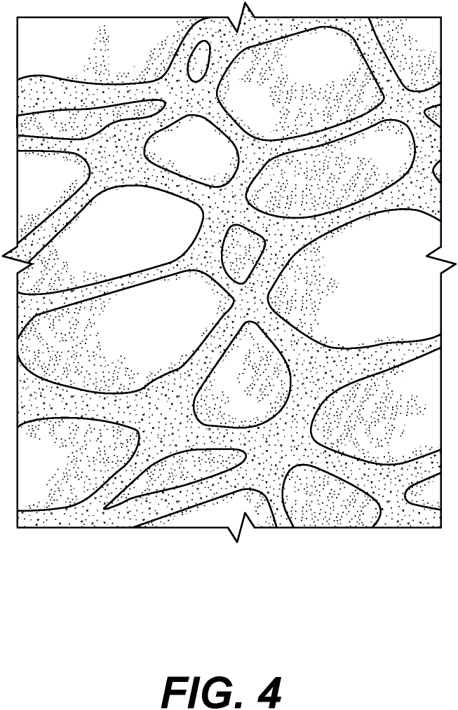

The non-viscoelastic foam may be formed having air capsules formed therein, such as microscopic air capsules formed within the non-viscoelastic foam that may be created during manufacturing of the non-viscoelastic foam. For example, FIG. 4 shows a structural view of a suitable non-viscoelastic foam having a plurality of air capsules formed therein that enable the foam material to have fast recovery characteristics and other suitable traits of the non-viscoelastic foam described herein.

The operating table anti-skid pad 10 may be resiliently deformable to provide comfort to a patient and reduce pressure on portions of the patient while retaining characteristics that permit the operating table anti-skid pad 10 to be compressed, folded, or otherwise manipulated during transportation and storage of the operating table anti-skid pad 10. The operating table anti-skid pad 10 has a substantially high rate of recovery, thereby permitting the operating table anti-skid pad 10 to rapidly recover from being folded, rolled, or otherwise compressed prior to use. A suitable non-viscoelastic foam of the operating table anti-skid pad 10 may be such that a body weight of a patient is more evenly distributed across a surface of the operating table anti-skid pad 10 to reduce occurrence of pressure points that may cause discomfort for a patient. For the pad 10 shown in FIGS. 1-3, the body is made from a material having an instantaneous rate of recovery, meaning there was no separation between the material and the indenter foot during the upward movement during a recovery time test per ASTM D3574-17, Test M (2023), as discussed below. However, in some implementations, the material has a rate of recovery of 1.0 seconds or less. In some implementations, the material has a rate of recovery of 1.5 seconds or less. In some implementations, the material has a rate of recovery of 2.0 seconds or less.

Table 1 shows recovery time testing results of three example samples of materials. Sample 1 includes a viscoelastic/memory foam material, Sample 2 includes a non-viscoelastic foam material, and Sample 3 includes a gel-visco material. As noted above, the recovery time testing was performed per ASTM D3574-17, Test M (2023). In the testing, a 15 × 15 × 4 inch specimen was indented to 75% of its initial thickness under a 4.5 N preload at a speed of 1000 mm/minute. After a 60 second dwell time, the indenter foot was returned to a 5% deflection at 1000 mm/minute and a stopwatch was started immediately upon initiating the upward movement of the indenter. The stopwatch was stopped as soon as the foam recovered to a 4.5 N preload on the indenter. If there was no separation between the foam and the indenter foot during the upward movement of the indenter foot, the recovery time was instantaneous under this method. As with all of the testing discussed below, the materials were all conditioned for 24 hours minimum at 23+/-2° C. and 50+/-5% relative humidity prior to testing.

TABLE 1

| | | |
|---|---|---|
| Recovery Time | Units | Result |
| Sample 1 | Sec | 7.0 |
| Sample 2 | Sec | Instantaneous |
| Sample 3 | Sec | Instantaneous |

The body 12 of the operating table anti-skid pad 10 may advantageously have a high enough static coefficient to prevent substantial sliding or movement of a patient on the pad 10 or substantial sliding or movement of the pad 10 on the operating table during a procedure. For the pad 10 shown in FIGS. 1-3, the body 12 is made from a material with a static coefficient of friction of 1.1 or greater. However, in some implementations, the static coefficient of the material is 1.0 or greater. In some implementations, the static coefficient of friction of the material is 0.9 or greater. In some implementations, the material has a static coefficient of friction between the material and the operating tabletop to prevent the pad from moving relative to the operating table when used in the Trendelenburg position. In some implementations, the material has a static coefficient of friction between the material and the patient to prevent the patient from moving relative to the pad when used in the Trendelenburg position.

Table 2 shows coefficient of friction testing results of the three example samples of materials from Table 1. The coefficient of friction testing was performed per ASTM D1894 (2023). The coefficient of friction of a material is the measure of the sliding resistance of a material over another material. A universal testing machine equipped with a load cell in its upper crosshead was used to pull a sled weighing 200 ± 5 g at a uniform surface speed of 150 mm/minute across a polished plane for a distance of approximately 130 mm. Each reading shown in Table 2 is the average of three pulls. In this test set up, the plane material (Purple flexible Vinyl material) was common to all tests and the sled surface contained the three different sample foam materials. Note that the sled weight varied slightly over the 200 ± 5 g requirement due to heavy viscoelastic foam materials. Temperature at the test was 23.3° C.

TABLE 2

| Base Material Tested Vinyl material (Purple) | Max. Reading (g) | Avg. Reading (g) | Sled Weight (g) | Static coefficient of Friction, µs | Kinetic coefficient of Friction, µk |
|---|---|---|---|---|---|
| Sample 1 | 309.3 | 189.8 | 209.6 | 1.476 | 0.906 |
| Sample 2 | 221.7 | 195.3 | 205.6 | 1.078 | 0.950 |
| Sample 3 | 268.0 | 202.6 | 206.5 | 1.298 | 0.981 |

Although the samples shown in Table 2 include various coefficient of friction testing results, in some implementations, the material has a static coefficient of friction of up to 1.1 or less, 1.2 or less, 1.3 or less, or 1.4 or less.

Suitable foams may have an indentation force deflection (IFD) measurement performed according to known testing standards that is the same or substantially similar to an IFD of a viscoelastic foam material while retaining fast recovery and other characteristics described herein. Similarly, suitable non-viscoelastic foams described herein may have pressure mapping characteristics such that average and peak pressures of a patient lying on the operating table anti-skid pad 10 may be substantially similar to average and peak pressures for a viscoelastic foam.

Tables 3 and 4 show IFD testing results of the three example samples of materials from Table 1. The IFD testing was performed per ASTM D3574-Test B1 (2023) at 25%, 65%, and R25%. The testing shown in Table 3 was performed on four 1-inch thick sheets of the material that were plied to form a 4 inch thick piece of material. The testing shown in Table 4 was performed on a single 1-inch thick sheets of the material.

TABLE 3

| Indentation Force Deflection | 25% Deflection (lb_{f}) | 65% Deflection (lb_{f}) | R25% Deflection (lb_{f}) | Support Factor (SF) 65%/25% | Recovery R25%/25% (%) |
|---|---|---|---|---|---|
| Sample 1 | 8.8 | 21.9 | 7.8 | 2.49 | 88.4 |
| Sample 2 | 13.2 | 23.6 | 11.3 | 1.79 | 85.8 |
| Sample 3 | 8.7 | 21.7 | 7.5 | 2.51 | 86.5 |

TABLE 4

| Indentation Force Deflection | 25% Deflection (lb_{f}) | 65% Deflection (lb_{f}) | R25% Deflection (lb_{f}) | Support Factor (SF) 65%/25% | Recovery R25%/25% (%) |
|---|---|---|---|---|---|
| Sample 1 | 7.0 | 16.8 | 6.4 | 2.41 | 91.4 |
| Sample 2 | 9.9 | 15.6 | 8.6 | 1.57 | 86.8 |
| Sample 3 | 6.8 | 16.7 | 6.0 | 2.45 | 88.5 |

Table 5 shows density testing results of the three example samples of materials from Table 1. The density testing was performed per ASTM D3574-17, Test A (2023). The testing was performed on 50 mm × 50 mm × 25 mm sheets of the materials.

TABLE 5

| Density, Core | Result Specimen 1 (lb/ft³) | Result Specimen 2 (lb/ft³) | Result Specimen 3 (lb/ft³) | Average Result (lb/ft³) |
|---|---|---|---|---|
| Sample 1 | 6.24 | 6.23 | 6.22 | 6.23 |
| Sample 2 | 2.91 | 2.92 | 2.93 | 2.92 |
| Sample 3 | 3.92 | 3.91 | 3.90 | 3.91 |

Table 6 shows air flow testing results of the three example samples of materials from Table 1. The density testing was performed per ASTM D3574-17, Test G (2023). The testing was performed on 50 mm × 50 mm × 25 mm sheets of the materials.

TABLE 6

| Air Flow | Result Specimen 1 (CFM) | Result Specimen 2 (CFM) | Result Specimen 3 (CFM) | Average Result (CFM) |
|---|---|---|---|---|
| Sample 1 | 0.90 | 0.85 | 0.85 | 0.87 |
| Sample 2 | 0.36 | 0.35 | 0.37 | 0.36 |
| Sample 3 | 2.84 | 2.72 | 2.74 | 2.77 |

Although the samples shown in Table 6 include various air flow testing results, in some implementations, the material has an air flow of up to 3.0 CFM or less, 3.25 CFM or less, 3.5 CFM or less, 3.75 CFM or less, 4.0 CFM or less, 4.25 CFM or less, 4.33 CFM or less, or 4.5 CFM or less.

Table 7 shows resilience (ball rebound) testing results of the three example samples of materials from Table 1. The resilience testing was performed per ASTM D3574-17, Test H (2023). The testing was performed on two 50 mm × 50 mm × 25 mm sheets of the same material that were plied to form a 50 mm × 50 mm × 50 mm sheet.

TABLE 7

| Resilience (Ball Rebound) | Result Specimen 1 (%) | Result Specimen 2 (%) | Result Specimen 3 (%) | Average Result (%) |
|---|---|---|---|---|
| Sample 1 | <1 | <1 | <1 | <1 |
| Sample 2 | 15.6 | 15.1 | 16.5 | 15.7 |
| Sample 3 | 11.9 | 11.7 | 12.7 | 12.1 |

Although the samples shown in Table 7 include various resilience (ball rebound) testing results, in some implementations, the material has a resilience (ball rebound) of up to 16% or less, 18% or less, 20% or less, 22% or less, 24% or less, 26% or less, 28% or less, 30% or less, 32% or less, 34% or less, 36% or less, or 38% or less.

Table 8 shows tensile strength testing results of the three example samples of materials from Table 1. The tensile strength testing was performed per ASTM D3574-17, Test E (2023). The testing was performed on a 0.5 inch thick sheet of the material.

TABLE 8

| Tensile Strength | Result Specimen 1 (PSI) | Result Specimen 2 (PSI) | Result Specimen 3 (PSI) | Average Result (PSI) |
|---|---|---|---|---|
| Sample 1 | 4.77 | 4.58 | 4.47 | 4.61 |
| Sample 2 | 5.53 | 5.58 | 5.93 | 5.68 |
| Sample 3 | 8.58 | 8.63 | 8.76 | 8.66 |

Although the samples shown in Table 8 include various tensile strength testing results, in some implementations, the material has a tensile strength of up to 6.0 PSI or less, 6.5 PSI or less, 7.0 PSI or less, 7.5 PSI or less, 8.0 PSI or less, 8.5 PSI or less, 9.0 PSI or less, 9.5 PSI or less, 10.0 PSI or less, 10.5 PSI or less, 11.0 PSI or less, 11.5 PSI or less, 12.0 PSI or less, 12.5 PSI or less, 13.0 PSI or less, 13.5 PSI or less, 14.0 PSI or less, 14.5 PSI or less, 15.0 PSI or less, 15.5 PSI or less, 16.0 PSI or less, 16.5 PSI or less, or 17.0 PSI or less.

Table 8 shows elongation testing results of the three example samples of materials from Table 1. The elongation testing was also performed per ASTM D3574-17, Test E (2023). Like with the tensile strength testing above, the elongation testing was performed on a 0.5 inch thick sheet of the material.

TABLE 9

| Elongation | Result Specimen 1 (%) | Result Specimen 2 (%) | Result Specimen 3 (%) | Average Result (%) |
|---|---|---|---|---|
| Sample 1 | 195 | 214 | 193 | 201 |
| Sample 2 | 135 | 169 | 155 | 153 |
| Sample 3 | 188 | 209 | 200 | 199 |

In contrast with viscoelastic foam, embodiments of the operating table anti-skid pad 10 described herein provide for fast recovery after deformation of the operating table anti-skid pad 10, thereby allowing the operating table anti-skid pad 10 to be deformed or compressed for packaging and transportation. In contrast with traditional foams, embodiments of the non-viscoelastic foam described herein are such that the operating table anti-skid pad 10 may have a coefficient of friction suitable to substantially prevent sliding of the operating table anti-skid pad 10 on an operating table, particularly when a patient is resting thereon. Further, the operating table anti-skid pad 10 may effectively reduce retention of heat from a body of a patient and be pliable at a wide range of effective temperatures.

Embodiments of the operating table anti-skid pad 10 described herein may be used during procedures in which a patient may be inclined, declined, or angled on an operating table or other surface. A coefficient of friction of the non-viscoelastic foam of the operating table anti-skid pad 10 may substantially prevent the patient and the operating table anti-skid pad 10 from sliding. A high rate of recovery of the non-viscoelastic foam, in contrast to viscoelastic foam, may provide greater support of a patient positioned on the operating table anti-skid pad 10 and to rapidly accommodate changes in position of the patient. The operating table anti-skid pad 10 may first be located on an operating table or other surface, and a patient may then be placed on top of the operating table anti-skid pad 10 prior to inclining, declining, or angling of the patient.

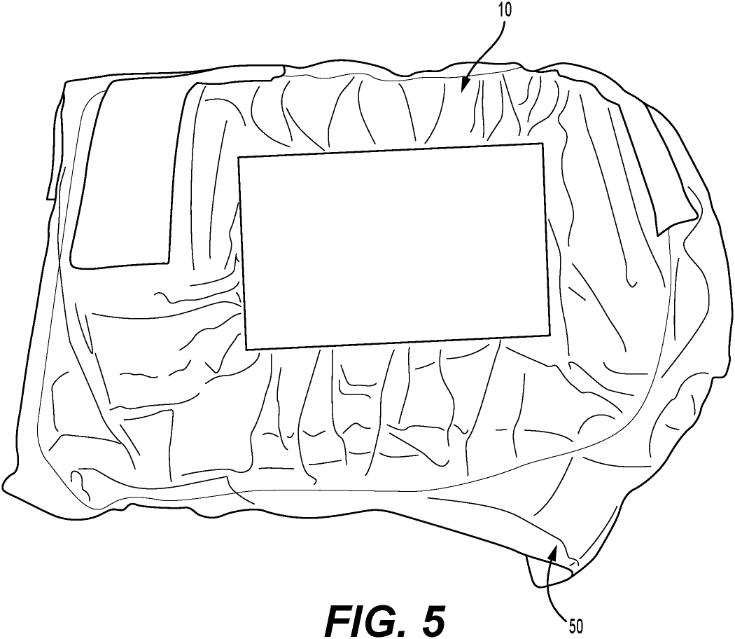

The operating table anti-skid pad 10 may be disposable and may be packaged such that the operating table anti-skid pad 10 is compressed prior to transportation and use of the operating table anti-skid pad 10. For example, the operating table anti-skid pad 10 may be folded across a length and width of the operating table anti-skid pad 10 such that an area of the operating table anti-skid pad 10 is reduced during transportation and storage of the operating table anti-skid pad 10. The operating table anti-skid pad 10 may otherwise be rolled or compressed to decrease a size of the operating table anti-skid pad 10 prior to use, as shown for example in FIG. 5. The pad may be compressed under negative pressure relative to atmospheric pressure within a vacuum sealed package 50. The operating table anti-skid pad 10 may then be released from packaging 50 or otherwise decompressed such that the operating table anti-skid pad 10 rapidly returns to a non-deformed shape prior to use.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. An operating table anti-skid pad, the pad comprising:
a body having a longitudinal axis, a first edge, a second edge spaced apart and opposite from the first edge, a third edge extending between the first edge and the second edge, and a fourth edge opposite and spaced apart from the third edge;
wherein the body comprises non-viscoelastic, resiliently deformable material having a rate of recovery of 2.0 seconds or less.

2. The pad of claim 1, wherein the rate of recovery is 1.5 seconds or less.

3. The pad of claim 2, wherein the rate of recovery is 1.0 seconds or less.

4. The pad of claim 1, wherein the material comprises a polyol.

5. The pad of claim 4, wherein the polyol comprises a high molecular weight polyol.

6. The pad of claim 1, wherein the material is a non-viscoelastic foam.

7. The pad of claim 1, wherein the body has a glass transition temperature of less than -20° C.

8. The pad of claim 7, wherein the glass transition temperature is less than -50° C.

9. The pad of claim 1, wherein a static coefficient of friction of the material is 0.9 or greater.

10. The pad of claim 9, wherein the static coefficient of the material is 1.0 or greater.

11. The pad of claim 10, wherein the static coefficient of friction of the material is 1.1 or greater.

12. The pad of claim 1, wherein the body further has a first surface extending between the first edge, the second edge, the third edge, and the fourth edge and a second surface opposite and spaced apart from the first surface, wherein the body has a thickness as measured between the first surface and the second surface, wherein the thickness of the body is 4 inches or less.

13. The pad of claim 12, wherein the thickness is from about 1 inch to about 2 inches.

14. The pad of claim 12, wherein the body has a variable the thickness.

15. The pad of claim 1, wherein the body further has a first surface extending between the first edge, the second edge, the third edge, and the fourth edge and a second surface opposite and spaced apart from the first surface, wherein the first surface of the body defines a cutout adjacent the first edge and extending toward the second surface.

16. The pad of claim 15, wherein the cutout is at least partially defined by the first edge.

17. The pad of claim 15, wherein the cutout is semi-circular or circular as viewed in a plane defined by the first surface.

18. The pad of claim 1, further comprising one or more straps extending from the third edge or the fourth edge.

19. The pad of claim 1, further comprising one or more arm tucking wings extending from the third edge or the fourth edge, wherein the one or more arm tucking wings are disposed closer to the first edge than to the second edge.

20. A system comprising:
a pad of claim 1; and
a vacuum sealed package,
wherein the pad is disposed within the vacuum sealed package, the pad being compressed by a negative pressure within the vacuum sealed package.
